(19) United States
(12) Patent Application Publication
(10) Pub. No.: US 2024/0197581 A1
(43) Pub. Date: Jun. 20, 2024

(54) HAND DISINFECTANT AS ALCOHOLIC FOAM

(71) Applicant: Bode Chemie GmbH, Hamburg (DE)

(72) Inventors: Jana Kuboteit, Seevetal (DE); Sven Eggerstedt, Hamburg (DE); Felix Krause-Kyora, Hamburg (DE)

(21) Appl. No.: 18/545,347

(22) Filed: Dec. 19, 2023

(30) Foreign Application Priority Data
Dec. 20, 2022 (DE) 102022134202.6

(51) Int. Cl. A61K 8/04; A61K 8/31; A61K 8/34; A61K 8/37; A61K 8/891; A61Q 17/00

(52) U.S. Cl. CPC A61K 8/046; A61K 8/31; A61K 8/34; A61K 8/37; A61K 8/375; A61K 8/891; A61Q 17/005; A61K 2800/52

(57) ABSTRACT

The invention relates to a foamable disinfectant containing at least one monohydric C2 to C4 alcohol, at least one silicone surfactant and foam stabilizers, wherein the disinfectant is present as a foam. The highly alcoholic solution of the disinfectant according to the invention is a hand disinfectant and is readily foamable by means of a foam applicator. In addition, the disinfectant exhibits good foam stability despite the very high alcohol content. The high alcohol content leads, as expected, to good antimicrobial effectiveness.

The invention relates to a foaming disinfectant comprising at least one monohydric alcohol having 2 to 4 carbon atoms, at least one silicone surfactant and foam stabilizers. The present invention further relates to the use of the disinfectant for hand disinfection.

In many settings, hand disinfection is necessary and in some cases even prescribed by law, for example in hospitals, medical practices, the food industry and the pharmaceutical industry. This commonly involves the use of preparations having a high content of lower monohydric alcohols such as ethanol, 1-propanol or 2-propanol owing to the good antibacterial effectiveness. When using alcoholic preparations for hand disinfection, hands and forearms are rubbed with the disinfectant and kept moist for half a minute to five minutes. In this way, hospital staff, for example, perform disinfection on the relevant areas of skin. There are various forms of application for hand disinfectants, with use being made of not only the conventional solution or emulsion, but also gels or foams.

Alcohols have foam-destabilizing properties. Therefore, it is necessary to use additional foam stabilizers in disinfectant foams. Foamable alcoholic hand disinfectants are known from the prior art. The foam stabilizers that are customarily used, for example cyclomethicone, are coming under increasing criticism because they are classified as being of concern by the competent European authorities, such as the European Chemicals Agency (ECHA).

EP 3 187 165 A1 describes a foamable hand disinfectant containing not only alcohol and silicone surfactants, but also aromatic alcohols and additionally fluorosurfactants as foaming agents. Despite a high alcohol content, the addition of the fluorosurfactants achieves stable foams.

It is an object of the present invention to provide a highly alcoholic composition which does not contain any foam stabilizers classified to be of concern and can thus also meet more stringent regulatory requirements. Moreover, the highly alcoholic disinfectant is to be foamable by means of a foam applicator and has to exhibit both good foam stability and good antimicrobial effectiveness.

These objects are achieved by an alcoholic, foaming disinfectant as claimed in claim 1 and the use as claimed in claims 12 and 13.

Preferred embodiments of the invention with further advantages are subject matter of the subclaims or described below.

The alcoholic, foaming disinfectant according to the invention comprises
at least one monohydric alcohol having 2 to 4 carbon atoms,
at least one silicone surfactant,
at least three foam stabilizers selected from the group consisting of
   i. trialkyl citrates, wherein the alkyl residue is a linear alkyl residue having 2 to 5 carbon atoms,
   ii. alkyl ether carboxylates,
   iii. a mixture of neopentyl glycol diheptanoate and isododecane,
   iv. triheptanoin,
optionally further additives and
water.

The alcoholic disinfectant according to the invention is foaming, i.e., it can form foam under mechanical stress, for example in a foam applicator, despite the alcohol content.

The disinfectant preferably contains at least three foam stabilizers selected from the group consisting of
i. trialkyl citrate, preferably triethyl citrate or tributyl citrate,
ii. potassium laureth-4 carboxylate or sodium laureth-5 carboxylate,
iii. a mixture of neopentyl glycol diheptanoate and isododecane, preferably a mixture of 28% to 32% by weight of neopentyl glycol diheptanoate and 68% to 72% by weight of isododecane, and
iv. triheptanoin.

The foam stabilizer of group i. trialkyl citrate is preferably triethyl citrate or tributyl citrate. Triethyl citrate (CAS No. 77-93-0) is also referred to as citric acid triethyl ester or, according to IUPAC, as triethyl 2-hydroxypropane-1,2,3-tricarboxylate. Tributyl citrate (CAS No. 77-94-1) is also referred to as citric acid tri-n-butyl ester or, according to IUPAC, as 1,2,3-tributyl 2-hydroxypropane-1,2,3-tricarboxylate.

The foam stabilizer of group ii. alkyl ether carboxylate is preferably potassium laureth-4 carboxylate (CAS No. 33939-64-9; IUPAC name poly(oxy-1,2-ethanediyl), alpha-(carboxymethyl)-omega-(dodecyloxy), sodium salt) or sodium laureth-5 carboxylate (CAS No. 38975-03-0; IUPAC name poly(oxy-1,2-ethanediyl), alpha-dodecyl-omega-carboxymethoxy, sodium salt).

The foam stabilizer of group iii. is preferably a mixture of 28% to 32% by weight of neopentyl glycol diheptanoate and 68% to 72% by weight of isododecane, as sold for example under the name LEXFEEL® D4.

The foam stabilizer iv. is triheptanoin (CAS No. 620-67-7), which is referred to as propane-1,2,3-triyl trisheptanoate according to IUPAC.

The foam stabilizers are preferably a mixture of triethyl citrate or tributyl citrate with potassium laureth-4 carboxylate or sodium laureth-5 carboxylate and at least one third foam stabilizer from groups iii. and iv.

The disinfectant contains the foam stabilizers preferably in amounts of
i. 0.1% to 1% by weight of trialkyl citrate,
ii. 0.1% to 1% by weight of alkali metal laureth carboxylate,
iii. 0.1% to 1% by weight of a mixture of neopentyl glycol diheptanoate and isododecane, and/or
iv. 0.1% to 1% by weight of triheptanoin,
if they are present. The amounts specified are based in each case on the total weight of the unfoamed disinfectant.

The disinfectant may also contain cocobetaine as an additional or alternative foam stabilizer. In this case, the disinfectant comprises, besides the alcohol, the silicone surfactant, the optional additives and the water, at least three foam stabilizers selected from the group consisting of
i. trialkyl citrates, wherein the alkyl residue is a linear alkyl residue having 2 to 5 carbon atoms,
ii. alkyl ether carboxylates,
iii. a mixture of neopentyl glycol diheptanoate and isododecane,
iv. triheptanoin,
v. cocobetaine.

Cocobetaine (CAS No.: 68424-94-2) is also referred to as coco alkyldimethyl betaine or coco dimethyl glycine and is for example obtainable under the trade name Amphosol@ DM. If the foam stabilizer v. is present, it is preferably present in an amount of 0.1% to 1% by weight, based on the total weight of the unfoamed disinfectant. The foam stabilizer v. is preferably used in a mixture with triethyl citrate or tributyl citrate and at least one third foam stabilizer from groups iii. and iv. Here, cocobetaine is thus in particular a possible alternative to the alkyl ether carboxylates.

The monohydric alcohol having 2 to 4 carbon atoms is preferably ethanol, 1-propanol, 2-propanol or a mixture thereof, preferably ethanol. Preferably, the disinfectant contains 75% by weight to 90% by weight, preferably 75-87% by weight, of the monohydric alcohol, based on the total weight of the unfoamed disinfectant. Apart from the alcohol, the hand disinfectant according to the invention preferably does not contain any other antimicrobial active ingredients.

Preferably, the disinfectant contains at least one silicone surfactant selected from the group consisting of PEG/PPG dimethicone and PEG dimethicone. Particularly preferably, the silicone surfactant is selected from the group consisting of bis-PEG/PPG-20/20 dimethicone, PEG/PPG-20/6 dimethicone, PEG/PPG-4/12 dimethicone, PEG/PPG-14/4 dimethicone, PEG-14 dimethicone, a mixture of bis-PEG/PPG-20/5 PEG/PPG-20/5 dimethicone, methoxy PEG/PPG-25/4 dimethicone and caprylic/capric triglycerides, PEG-17 dimethicone, PEG/PPG-18/18 dimethicone, bis-PEG-18 methyl ether dimethyl silane, PEG/PPG-25/25 dimethicone, and cetyl PEG/PPG-10/1 dimethicone. In particular, the silicone surfactant is bis-PEG/PPG-20/20 dimethicone.

The at least one silicone surfactant is preferably present in the disinfectant to an extent of 0.1% by weight to 5% by weight, preferably 0.5% to 2% by weight, based on the total weight of the unfoamed disinfectant.

In one embodiment, the alcoholic, foaming disinfectant comprises
75% to 90% by weight of at least one monohydric alcohol,
0.5% to 2% by weight of at least one silicone surfactant,
1.0% to 2.0% by weight of foam stabilizers selected from groups i., ii., iii and/or iv.,
0% to 3% by weight, preferably 0.15% to 2.5% by weight, of further additives and water,
based in each case on the total weight of the unfoamed disinfectant.

Preferably, the alcoholic, foaming disinfectant comprises
75% to 90% by weight of ethanol, 1-propanol, 2-propanol or a mixture thereof,
0.5% to 2% by weight of PEG/PPG dimethicone and/or PEG dimethicone,
1.0% to 2.0% by weight of a foam stabilizer mixture consisting of
   triethyl citrate or tributyl citrate,
   potassium laureth-4 carboxylate or sodium laureth-5 carboxylate
   and at least one third foam stabilizer from groups iii. and iv.,
0% to 3% by weight of further additives, preferably skin moisturizers,
   skincare agents, dyes and/or perfume, and
water,
based in each case on the total weight of the unfoamed disinfectant.

In one embodiment, the disinfectant consists of
75% to 90% by weight of at least one monohydric alcohol,
0.5% to 2% by weight of at least one silicone surfactant,
1.0% to 2.0% by weight of foam stabilizers,
0.1% to 1.0% by weight of glycerin or glycerin mixture,
0% to 2% by weight, preferably 0.1% to 2% by weight, of further additives and
water,
based in each case on the total weight of the unfoamed disinfectant, the components adding up to 100% by weight.

Preferably, the disinfectant contains glycerin or a glycerin mixture as a skincare agent. Glycerin or the glycerin mixture is preferably present in concentrations of 0.1% to 1% by weight if it is added. The disinfectant foam can contain further skincare agents, including natural moisturizing factors and care substances such as isopropyl myristate, tocopheryl acetate, jojoba esters, 1,3-propanediol, heptamethylnonane, bisabolol, panthenol and allantoin. In addition, further additives customarily used in alcoholic hand disinfectants, for example surfactants such as lauryldimethylamine or lauric diethanolamide, can be present.

The foaming, alcoholic disinfectant according to the invention is used for hand disinfection. Hand disinfection is understood to mean a process which achieves the greatest possible reduction in the transient microbial hand flora in a short period of time, so that transmission of microbial contamination to third parties by the hands is avoided. Depending on whether the hand disinfection is hygienic hand disinfection or surgical hand disinfection, the exposure time for the hand disinfectant is half a minute to one minute (applicable for hygienic hand disinfection) or one minute to five minutes (applicable for surgical hand disinfection), during which the hands are kept moist. After successful hand disinfection, the hands can be regarded as hygienically safe. Alcoholic compositions having a high alcohol content, such as the hand disinfectant according to the invention, have the advantage that they exhibit comprehensive antimicrobial effectiveness after just a short exposure time of only 0.25 to 1 minute in suspension tests according to European standards, for example EN13727, EN13624, EN14476.

The foaming disinfectant contains at least three foam stabilizers for foam stabilization. The substances which are used as foam stabilizers were previously only known as plasticizers, but not described with a foam-stabilizing effect. Particularly the use of alkyl ether carboxylates, in combination with the citric acid triesters and at least one further new foam stabilizer, leads to a distinctly better consistency and stability of the alcoholic foam. All the substances from groups i., ii., iii. and iv. contribute to foam stabilization.

At the same time, the touch and feel of the alcoholic foam, i.e., the skin sensation, is important. The disinfectant exhibits both a good foaming behavior and, at the same time, a good touch and feel, since it does not stick and has a good skin sensation.

For use as a hand disinfectant, the alcoholic foam must be initially stable, i.e., it must not dissolve at once and not drip during application. However, the foam must not be too stable and must not remain a foam for too long, since it must be spread over the hand. This means that the foam must be stable during application, but must dissolve and spread over the hand within 15-30 seconds. The foam disappears when spread over the hand.

The highly alcoholic solution of the disinfectant according to the invention is readily foamable by means of a foam applicator, in particular a pressureless foam applicator. Suitable, for example, are foam applicators which, depending on the embodiment, pump between 0.75 ml, 1.0 ml, 1.2 ml and 1.5 ml of liquid per stroke, corresponding to a foam dose of 9 ml, 10 ml or 12 ml. For example, foam applicators which foam up 1 ml of liquid to form about 10 ml of foam are used.

The disinfectant according to the invention is applied in the form of an alcoholic foam which is based on a highly alcoholic aqueous system. The application is preferably carried out by means of a pressureless foam applicator. The foam applicator is preferably a foam dispenser or a foam pump.

A high alcohol content is desirable in order to achieve the necessary efficacy with only a small amount (about 3 ml). Reference is made here to the standard EN 1500, “Hygienic Handrub”. In the case of a high alcohol content, two to three strokes from the foam applicator are generally sufficient for obtaining the required amount.

The disinfectant according to the invention exhibits good foam stability despite the typically very high content of alcohol. The high alcohol content leads, as expected, to good antimicrobial effectiveness. Effectiveness in accordance with EN1500 (Hygienic Handrub) and EN14476 (Virucidal Effectiveness) is demonstrated with an application time of 30 s. The disinfectant therefore has good antimicrobial effectiveness and forms a stable highly alcoholic foam. Moreover, the foam stabilizers used are environmentally friendly and skin-friendly.

### EXAMPLES

The invention will be more particularly elucidated on the basis of the examples.

The foamable solutions are prepared in a simple mixing process. The liquid components alcohol and water are charged in the first step. The further ingredients are then added successively while stirring and are stirred until there is a clear solution.

What were prepared were disinfectants according to the invention in accordance with the formulations in Table 1.

TABLE 1
Formulations of the prepared disinfectants
according to the invention

| | Example No. | | | | | | | |
|---|---|---|---|---|---|---|---|---|
| | 1 | 2 | 3 | 4 | 5 | 6 | 7 | 8 |
| Component | [%] | [%] | [%] | [%] | [%] | [%] | [%] | [%] |
| Ethanol 99%, | 85.86 | 85.86 | 85.86 | 80.81 | — | 85.86 | 85.86 | 85.86 |
| denatured | | | | | | | | |
| with 2- | | | | | | | | |
| butanone | | | | | | | | |
| 2-Propanol | — | — | — | — | 45.00 | — | — | — |
| 1-Propanol | — | — | — | — | 30.00 | — | — | — |
| Bis-PEG/ | 1.00 | 1.00 | 1.00 | 1.00 | 1.00 | 1.00 | 1.00 | 1.00 |
| PPG-20/20 | | | | | | | | |
| dimethicone | | | | | | | | |
| Triethyl citrate | 0.50 | 0.50 | 0.50 | 0.50 | 0.50 | — | 0.50 | — |
| Tributyl citrate | — | — | — | — | — | 0.50 | — | 0.50 |
| Potassium | 0.40 | 0.50 | 0.98 | 0.40 | 0.40 | 0.40 | — | — |
| laureth-4 | | | | | | | | |
| carboxylate | | | | | | | | |
| Sodium | — | — | — | — | — | — | 0.40 | 0.40 |
| laureth-5 | | | | | | | | |
| carboxylate | | | | | | | | |
| LexFeel D4* | — | 0.40 | — | 0.40 | — | — | — | — |
| Triheptanoin | 0.40 | — | 0.40 | — | 0.40 | 0.40 | 0.40 | 0.40 |
| Isopropyl | 0.75 | 0.75 | 0.75 | 0.75 | 0.75 | 0.75 | 0.75 | 0.75 |
| myristate | | | | | | | | |
| Glycerin 85% | 0.50 | — | — | 0.50 | 0.50 | 0.50 | 0.50 | 0.50 |
| Hydagen | — | 0.40 | 0.40 | — | — | — | — | — |
| Aquaporin* | | | | | | | | |
| Floraester | 0.1 | 0.1 | 0.1 | 0.1 | 0.1 | 0.1 | 0.1 | 0.1 |
| K-20W | | | | | | | | |
| Jojoba* | | | | | | | | |
| Tocopherol | 0.1 | 0.1 | 0.1 | 0.1 | 0.1 | 0.1 | 0.1 | 0.1 |
| acetate | | | | | | | | |
| Purified water | 10.39 | 10.39 | 9.91 | 15.44 | 21.25 | 10.39 | 10.39 | 10.39 |
| (aqua | | | | | | | | |
| purificata) | | | | | | | | |

*LexFeel ® D4: mixture of neopentyl glycol diheptanoate and isododecane
*Hydagen Aquaporin ®: mixture of glyceryl glucosides and glycerin
*Floraester K-20W Jojoba ®: hydrolyzed jojoba esters with water

For comparison, disinfectants not according to the invention were prepared with only a single foam stabilizer (V1) or at most two foam stabilizers (V2). The formulations of the comparative examples are specified in Table 2.

TABLE 2
Formulations of the disinfectants prepared as comparison

| Example No. | V1 | V2 |
|---|---|---|
| Component | [%] | [%] |
| Ethanol 99%, denatured with 2- | 85.86 | 85.86 |
| butanone | | |
| Bis-PEG/PPG-20/20 dimethicone | 1.00 | 1.00 |
| Triethyl citrate | 1.00 | 0.50 |
| Potassium laureth-4 carboxylate | — | — |
| Sodium laureth-5 carboxylate | — | — |
| LexFeel ® D4 | — | 0.35 |
| Triheptanoin | — | — |
| Isopropyl myristate | 1.00 | 1.00 |
| Glycerin 85% | 0.50 | 0.50 |
| Hydagen Aquaporin ® | — | — |
| Floraester K-20W Jojoba ® | — | 0.10 |
| Tocopherol acetate | — | 0.10 |
| Lauramine oxide | — | 0.35 |
| Purified water (aqua purificata) | 10.64 | 10.14 |

With the disinfectants according to the comparative examples, it was not possible to produce a stable foam which can be applied to the hands. Disinfectant V1 with only a single foam stabilizer formed an unstable foam having large bubbles that sprayed during pumping with the foam applicator and that collapsed rapidly after application to the extent that it ran and dripped from the hand. Disinfectant V2 with two foam stabilizers formed a somewhat more stable foam, but also did not maintain foam stability for a sufficiently long time for application of the desired quality.

### Foamability

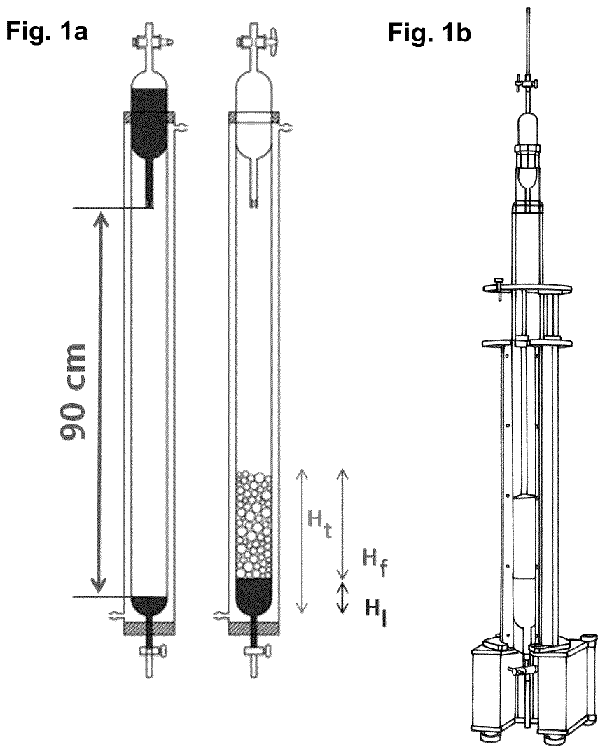

The foamability of Examples 1 to 8 was assessed by means of a Ross-Miles Foam Analyzer (RMFA) from Krüss in accordance with ASTM D1173-07 at 20° ° C.(see also FIG. 1).

FIG. 1a shows an illustration of a Ross-Miles foam analysis before foam formation (left) and after foam formation (right). The arrows indicate the total height Ht, the fluid height HI and the net foam height Hf as measured by the instrument. FIG. 1b shows an illustration of the KRÜSS RMFA for automatic foam analysis in accordance with Ross-Miles standard ASTM D 1173 07.

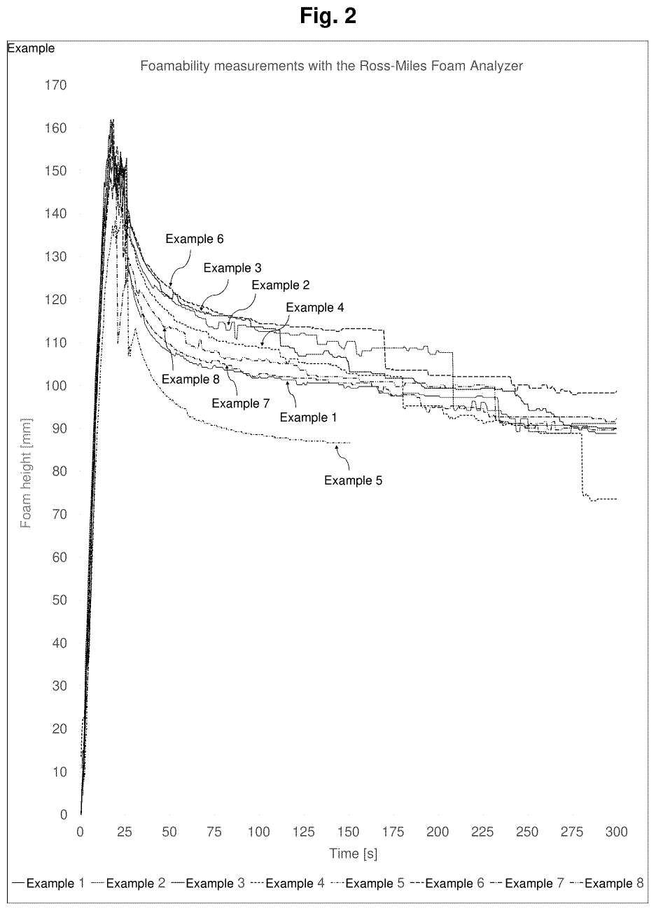

The results of the foamability measurements are shown in FIG. 2. The measurements were carried out over a period of 150 seconds in the case of Example 5 and over a period of 300 seconds in the case of the other examples. Between 0 and 25 seconds, the product foams by itself. This range is comparable to the foaming of the product by a foam pump. The application period is between 25 and not more than 75 seconds.

It is found that a distinctly higher foam height can be achieved in the case of the hand disinfectants according to the invention, i.e., that the compositions according to the invention foam better. Moreover, the foam is distinctly more stable over the interval of 0 seconds to 5 minutes in the case of the compositions according to the invention than in the case of the comparative formulations.

### Antimicrobial Effectiveness

The disinfectant was tested in respect of its antimicrobial effectiveness. Owing to the high alcohol content, there is bactericidal effectiveness in accordance with method EN 13727: 2015 and yeasticidal effectiveness in accordance with method EN 13624: 2021.

What were prepared were disinfectants according to the invention in accordance with the formulations in Table 3.

TABLE 3
Formulations of the prepared disinfectants
according to the invention

| Example No. | 9 | 10 | 11 |
|---|---|---|---|
| Component | [%] | [%] | [%] |
| Ethanol 99%, denatured with 2- | 85.86 | 85.86 | 85.86 |
| butanone | | | |
| Potassium laureth-4 carboxylate | 0.40 | 0.50 | 0.98 |
| Triethyl citrate | 0.50 | 0.50 | 0.50 |
| LexFeel ® D4 | — | 0.40 | — |
| Triheptanoin | 0.40 | — | 0.40 |
| Isopropyl myristate | 0.75 | 0.75 | 0.75 |
| Glycerin 85% | 0.50 | — | — |
| Hydagen Aquaporin ® | — | 0.40 | 0.40 |
| Bis-PEG/PPG-20/20 dimethicone | 1.00 | 1.00 | 1.00 |
| Floraester K-20W Jojoba ® | 0.10 | 0.10 | 0.10 |
| Tocopherol acetate | 0.10 | 0.10 | 0.10 |
| Purified water (aqua purificata) | 10.39 | 10.39 | 9.91 |

Virucidal effectiveness was measured by testing, inter alia, effectiveness against adenovirus. Effectiveness against adenovirus was tested in accordance with EN14476:2019 under clean conditions. Disinfectants according to Examples 9, 10 and 11 specified in Table 3 were tested. Sufficient effectiveness against adenovirus within 15 s was demonstrated for the disinfectant according to the invention. A reduction of RF ≥ 5.5 within 30 sec was achieved. Moreover, a very rapid effectiveness against poliovirus was also demonstrated for the disinfectant according to the invention. Effectiveness against poliovirus was tested in accordance with EN14476:2019 under dirty conditions. A reduction of RF ≥4 within 15 sec was achieved. The disinfectant can thus be advertised as “virucidal”.

Effectiveness in hygienic hand disinfection was tested in accordance with method EN 1500:2013. Suitability for hygienic hand disinfection is tested using the test microbe Escherichia coli. 3 ml of sample are rubbed in dry hands for 30 seconds. In accordance with EN 1500 (April 2013 edition, screening with 10 test subjects), the disinfectants according to the invention exhibited the necessary reduction in the test microbes.

Moreover, various antimicrobial effectivenesses were tested with a disinfectant according to the invention. Table 4 shows an overview of the microbial reductions achieved.

TABLE 4
Antimicrobial activities and test methods

| | | | Test | | |
|---|---|---|---|---|---|
| | | | concentration | Reduction | Reduction |
| Method | Microbe | Condition | [%] | time | factor |
| EN 1500 | Staphylococcus aureus | | | 30 s/3 mL | fulfilled |
| EN 14476: 2013 + | Modified vaccinia | clean | 80 | 15 s | ≥5.79 |
| A2: 2019 | Ankara (MVA) | | | | |
| EN 14476: 2013 + | Adenovirus | clean | 80 | 15 s | ≥5.61 |
| A2: 2019 | | | | | |
| EN 14476: 2013 + | Murine norovirus (MNV) | clean | 80 | 15 s | ≥5.18 |
| A2: 2019 | | | | | |
| EN 14476: 2013 + | Poliovirus | clean | 80 | 15 s | ≥4.18 |
| A2: 2019 | | | | | |
| EN 13727: 2012 + | Staphylococcus aureus | clean | 80 | 15 s | ≥5 |
| A2: 2015 | | | | | |
| | Enterococcus hirae | clean | 80 | 15 s | ≥5 |
| | Escherichia coli K12 | clean | 80 | 15 s | ≥5 |
| | Pseudomonas aeruginosa | clean | 80 | 15 s | ≥5 |
| EN 13624: 2021 | Candida albicans | clean | 80 | 15 s | ≥4 |
| EN 14348: 2005 | Mycobacterium terrae | clean | 80 | 15 s | ≥4 |
| EN 14348: 2005 | Mycobacterium avium | clean | 80 | 15 s | ≥4 |

It was demonstrated that the disinfectant according to the invention is effective against viruses, polioviruses, fungi, mycobacteria and also bacteria and achieves the required microbial reduction within 15 sec in each case. Therefore, it is possible to advertise the disinfectant as bactericidal, virucidal, levurocidal, mycobactericidal and tuberculocidal.

### Skin Compatibility

As part of a skin study, a disinfectant according to the invention was applied 20 times daily to 20 test subjects for 14 days. Skin hydration was measured by means of corneometry and capacitive contact imaging (CCI, Epsilon Model E100). Moreover, a visual assessment was carried out by a dermatologist before the first application and after the last application and the test subjects were surveyed on the product properties.

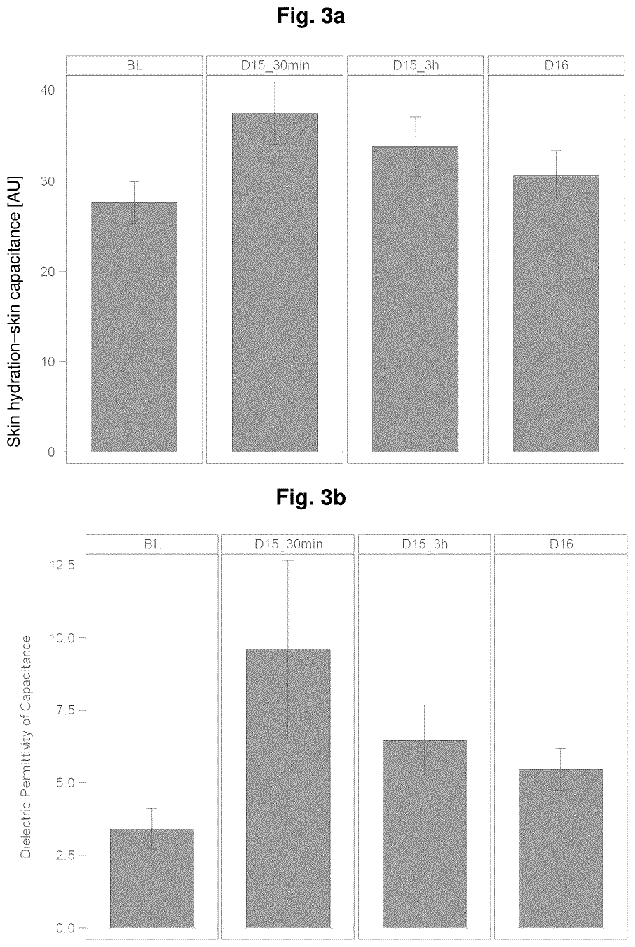

A significant increase in skin hydration was measured by means of corneometry and capacitive contact imaging (CCI). Thus, FIG. 3a shows the mean values of skin hydration-skin capacitance in arbitrary units (AU) measured by means of a corneometer as a bar chart with mean values and 95% confidence intervals of the raw data (n=19-20). D means day and D16 is day 16 after the start of the study. BL means baseline and is the value of the baseline without application of a product to the skin.

The following measured values are shown:
BL: Baseline assessment before the first application of the test product
Day 15_30 min: 30 minutes after the last application
Day 15_3 h: 3 hours after the last application
Day 16: 24 hours after the last application

FIG. 3b shows the dielectric permittivity of capacitance by capacitive contact imaging (CCI) as a bar chart with mean values and 95% confidence intervals of the raw data (n=19-20).

The skin compatibility assessment by the dermatologist showed a tendency toward a reduction in the dryness, redness and desquamation of the skin. This was also in line with the feeling of the test subjects.

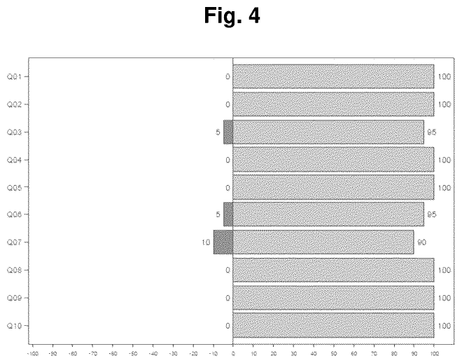

After the first application, the test subjects completed a questionnaire to rate the product, which reflects a very positive perception of the product. The evaluation of the questionnaire is shown in FIG. 4. The left-hand bars in the negative value range of the x-axis indicate disagreement, and the right-hand bars in the positive value range of the x-axis indicate agreement (in %). The following questions were asked, with the test subjects indicating “Agree” or “Disagree” in each case.
Question Q01: The test product gives a pleasant skin sensation
Question Q02: The test product provides skincare even during disinfection
Question Q03: The test product gives a smooth skin sensation
Question Q04: The test product gives a soft/supple skin sensation
Question Q05: The test product provides instant hydration
Question Q06: The test product can be spread comfortably
Question Q07: The test product is not sticky
Question Q08: The test product is better than the disinfectant that I normally use (only for individuals who normally use a disinfectant)
Question Q09: I would recommend the test product
Question Q10: I would buy the test product

It was found that the caring properties and general product properties of the hand disinfection foam tested were rated very positively by the test subjects after just a single application.

### Measurement Methods

Corneometry is a method for determining skin hydration. This involves determining the dielectric permittivity of the top layers of the skin with the aid of a sensor. The dielectric permittivity varies depending on the water content of the skin.

Capacitive contact imaging (CCI, Epsilon Model E100): In capacitive contact imaging, the dielectric permittivity of the top layers of the skin is determined with the aid of a sensor. The dielectric permittivity varies depending on the water content of the skin. The technology provides imaging data and skin hydration data at the same time.

The invention is not restricted to one of the embodiments described above, but may be modified in a variety of ways.

All the features and advantages that are apparent from the claims, the description and the drawings may be essential to the invention both individually and in a wide variety of combinations.

1. A foaming disinfectant comprising:
at least one monohydric alcohol having 2 to 4 carbon atoms,
at least one silicone surfactant,
at least three foam stabilizers selected from the group consisting of:
i. trialkyl citrate, wherein the alkyl residue is a linear alkyl residue having 2 to 5 carbon atoms,
ii. alkyl ether carboxylate,
iii. a mixture of neopentyl glycol diheptanoate and isododecane, and
iv. triheptanoin, and
water.

2. The disinfectant as claimed in claim 1, wherein the at least three foam stabilizers are selected from the group consisting of:
i. trialkyl citrate,
ii. potassium laureth-4 carboxylate or sodium laureth-5 carboxylate,
iii. a mixture of neopentyl glycol diheptanoate and isododecane, and
iv. triheptanoin.

3. The disinfectant as claimed in claim 2, wherein the foam stabilizers are:
i. triethyl citrate or tributyl citrate and
ii. potassium laureth-4 carboxylate or sodium laureth-5 carboxylate and at least one third foam stabilizer from groups iii. And iv.

4. The disinfectant as claimed in claim 2, wherein the disinfectant contains one or more foam stabilizers in amounts of
i. 0.1% to 1% by weight of trialkyl citrate,
ii. 0.1% to 1% by weight of alkali metal laureth carboxylate,
iii. 0.1% to 1% by weight of a mixture of neopentyl glycol diheptanoate and isododecane, or
iv. 0.1% to 1% by weight of triheptanoin,
based in each case on the total weight of the unfoamed disinfectant.

5. The disinfectant as claimed in claim 1, wherein the monohydric alcohol is ethanol, 1-propanol, 2-propanol or a mixture thereof.

6. The disinfectant as claimed in claim 1, wherein the disinfectant contains 75% by weight to 90% by weight, of alcohol, based on the total weight of the unfoamed disinfectant.

7. The disinfectant as claimed in claim 1, wherein the silicone surfactant is selected from the group consisting of PEG/PPG dimethicone and PEG dimethicone.

8. The disinfectant as claimed in claim 7, wherein the silicone surfactant is selected from the group consisting of bis-PEG/PPG-20/20 dimethicone, PEG/PPG-20/6 dimethicone, PEG/PPG-4/12 dimethicone, PEG/PPG-14/4 dimethicone, PEG-14 dimethicone, a mixture of bis-PEG/PPG-20/5 PEG/PPG-20/5 dimethicone, methoxy PEG/PPG-25/4 dimethicone and caprylic/capric triglycerides, PEG-17 dimethicone, PEG/PPG-18/18 dimethicone, bis-PEG-18 methyl ether dimethyl silane, PEG/PPG-25/25 dimethicone, and cetyl PEG/PPG-10/1 dimethicone.

9. The disinfectant as claimed in claim 1, wherein the disinfectant contains 0.1% by weight to 5% by weight, of silicone surfactant, based on the total weight of the unfoamed disinfectant.

10. The disinfectant as claimed in claim 1, wherein the disinfectant contains:
75% to 90% by weight of monohydric alcohol,
0.5% to 2% by weight of at least one silicone surfactant,
1.0% to 2.0% by weight of foam stabilizers,
0% to 3% by weight, of further additives and water,
based in each case on the total weight of the unfoamed disinfectant.

11. The disinfectant as claimed in claim 1, wherein the disinfectant contains:
75% to 90% by weight of monohydric alcohol,
0.5% to 2% by weight of at least one silicone surfactant,
1.0% to 2.0% by weight of foam stabilizers,
0.1% to 1.0% by weight of glycerin or glycerin mixture, and
0% to 2% by weight, of further additives and water,
based in each case on the total weight of the unfoamed disinfectant, the components adding up to 100% by weight.

12. The use of a disinfectant as claimed in claim 1 for hand disinfection.

13. A disinfectant as claimed in claim 1 for use in hand disinfection.

14. The disinfectant as claimed in claim 2, wherein the trialkyl citrate is triethyl citrate or tributyl citrate is a mixture of 28% to 32% by weight of neopentyl glycol diheptanoate and 68% to 72% by weight of isododecane.

15. The disinfectant as claimed in claim 2, wherein the mixture of neopentyl glycol diheptanoate and isododecane.

16. The disinfectant as claimed in claim 6, wherein the disinfectant contains preferably 75% to 87% by weight of alcohol.

17. The disinfectant as claimed in claim 9, wherein the disinfectant contains 0.5% to 2% by weight, of silicone surfactant based on the total weight of the unfoamed disinfectant.

18. The disinfectant as claimed in claim 10, wherein the further additives and water is 0.15% to 2.5% by weight.

19. The disinfectant as claimed in claim 11, wherein the further additives and water is 0.1% to 2% by weight.
